(19) United States
(12) Patent Application Publication
(10) Pub. No.: US 2023/0181174 A1
(43) Pub. Date: Jun. 15, 2023

(54) SPECIMEN RETRIEVAL DEVICE

(71) Applicant: Covidien LP, Mansfield, MA (US)

(72) Inventors: Garrett P. Ebersole, Hamden, CT (US); Justin J. Thomas, New Haven, CT (US); Jacob C. Baril, Norwalk, CT (US); Nicolette L. Roy, Windsor Locks, CT (US); Matthew A. Dinino, Newington, CT (US); Roy J. Pilletere, Middletown, CT (US); Saumya Banerjee, Southington, CT (US)

(21) Appl. No.: 17/984,293

(22) Filed: Nov. 10, 2022

Related U.S. Application Data
(60) Provisional application No. 63/289,673, filed on Dec. 15, 2021.

(51) Int. Cl. A61B 10/02; A61B 10/04

(52) U.S. Cl. CPC A61B 10/0233; A61B 10/04

(57) ABSTRACT

A specimen retrieval device for laparoscopic procedures includes a plurality of barbed threads, the barbed threads having an elongate body, a distal portion, a proximal portion, and barbs extending from the elongate body. The distal portions of the barbed threads are circumferentially affixed to a centering ring and a space is formed within a circumference of the barbed threads. A tissue specimen to be removed from a patient is placed within the space and the proximal portions of the barbed threads are pulled in a proximal direction to reduce the volume of the space within which the tissue specimen has been placed, so that the barbed threads come into contact with the tissue specimen, thereby securing the tissue specimen within the space. The specimen retrieval device may then be removed from the patient's body cavity.

### CROSS-REFERENCE TO RELATED APPLICATIONS

This application claims the benefit of the filing date of provisional U.S. Patent Application No. 63/289,673 filed on Dec. 15, 2021.

### FIELD

The disclosure relates to a surgical apparatus for use in minimally invasive surgical procedures, such as endoscopic and/or laparoscopic procedures and, more particularly, to a specimen retrieval device for removal of tissue from a patient.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy, and laryngobronchoscopy, just to name a few. In these procedures, trocars are utilized for creating incisions through which the endoscopic surgery is performed. Trocar tubes or cannula assemblies are extended into and left in place in the abdominal wall to provide access for endoscopic surgical tools. A camera or scope is inserted through a cannula assembly to permit the visual inspection and magnification of the body cavity. The surgeon can then perform diagnostic and/or therapeutic procedures at the surgical site with the aid of specialized instrumentation, such as forceps, specimen retrieval devices, cutters, applicators, and the like, which are designed to fit through additional cannulas.

Several minimally invasive surgical procedures require the bulk removal of body tissue or organs through a limited surgical opening. Improved devices for bulk removal of tissue remain desirable.

### SUMMARY

A specimen retrieval device of the present disclosure includes a plurality of barbed threads, each of the plurality of barbed threads having an elongate body, a distal portion, a proximal portion, and barbs extending from the elongate body. The specimen retrieval device also includes a centering ring, with the distal portions of the plurality of barbed threads circumferentially affixed to the centering ring.

In aspects, a space is formed within a circumference of the plurality of barbed threads.

In some aspects, the plurality of barbed threads includes from about 4 to about 12 barbed threads.

In other aspects, the plurality of barbed threads includes from about 6 to about 10 barbed threads.

The plurality of barbed threads may be formed of a biocompatible material selected from polymeric materials derived from lactones, carbonates, dioxanones, ethylene glycol, ethylene oxide, ester amides, γ-hydroxyvalerate, β-hydroxypropionate, alpha-hydroxy acid, hydroxybutyrates, poly (ortho esters), hydroxy alkanoates, polyurethanes, polyanhydrides, polybutesters, copolymers thereof, or combinations thereof.

The centering ring may be formed of a biocompatible material selected from polymeric materials derived from lactones, carbonates, dioxanones, ethylene glycol, ethylene oxide, ester amides, γ-hydroxyvalerate, β-hydroxypropionate, alpha-hydroxy acid, hydroxybutyrates, poly (ortho esters), hydroxy alkanoates, polyurethanes, polyanhydrides, polybutesters, copolymers thereof, or combinations thereof.

In aspects, the tips of the barbs face proximally.

Kits of the present disclosure include a specimen bag in combination with a specimen retrieval device including a plurality of barbed threads, each of the plurality of barbed threads having an elongate body, a distal portion, a proximal portion, and barbs extending from the elongate body. The specimen retrieval device also includes a centering ring, wherein the distal portions of the plurality of barbed threads are circumferentially affixed to the centering ring.

In aspects, the specimen bag includes a tubular configuration having an opening at a proximal portion of the specimen bag and a closed portion.

In other aspects, the specimen bag is formed of a biocompatible material selected from nylon, urethane, ripstop nylon or latex.

In aspects, the kit further includes at least one additional component selected from access ports, trocars, catheters, graspers, vacuum sources, or combinations thereof

In some aspects, the at least one additional component included with the kit is an access port.

Methods of the present disclosure include a method for removing tissue from a patient, which includes introducing a specimen retrieval device into a patient, the specimen retrieval device including a plurality of barbed threads, each of the plurality of barbed threads having an elongate body, a distal portion, a proximal portion, and barbs extending from the elongate body, and a centering ring, wherein the distal portions of the plurality of barbed threads are circumferentially affixed to the centering ring and a space is formed within a circumference of the plurality of barbed threads. A tissue specimen is then introduced into the space formed within the circumference of the plurality of barbed threads. The proximal portions of the plurality of barbed threads are pulled so that the barbed threads come into contact with the tissue specimen, thereby securing the tissue specimen within the space formed within the circumference of the plurality of barbed threads, and the specimen retrieval device is removed from the patient.

In aspects, pulling the proximal portions of the plurality of barbed threads occurs by pulling the proximal portions sequentially.

In other aspects, pulling the proximal portions of the plurality of barbed threads occurs by pulling the proximal portions simultaneously.

In aspects, the method includes introducing the specimen retrieval device into the patient through an access port.

In other aspects, the method includes inserting the specimen retrieval device into a specimen bag prior to removing the specimen retrieval device from the patient.

### BRIEF DESCRIPTION OF THE DRAWINGS

Various aspects of the disclosed specimen retrieval devices and methods are described herein below with reference to the drawings, wherein:

FIG. 1 is a side perspective view of a specimen retrieval device according to an aspect of the disclosure;

FIG. 2 is a side perspective view of the specimen retrieval device shown in FIG. 1 in use;

FIG. 3 is a side perspective view of the specimen retrieval device shown in FIG. 2, after placement of the specimen retrieval device within a specimen bag;

FIG. 4 is a side perspective view of usage of the specimen retrieval device of FIG. 3 within the specimen bag to remove a tissue specimen from a patient;

FIG. 5 is a side view perspective depicting removal of the specimen retrieval device and specimen bag shown in FIG. 3 from a patient's body; and

FIG. 6 is a side perspective view of the specimen retrieval device and specimen bag shown in FIG. 3 after removal from a patient's body.

### DETAILED DESCRIPTION

The present disclosure provides a specimen retrieval device for use in minimally invasive and other surgical procedures such as other reduced-access surgical procedures, partially-open surgical procedures, and open surgical procedures. As used herein, minimally invasive surgical procedures encompass laparoscopic procedures, arthroscopic procedures, and endoscopic procedures, and refer to procedures utilizing scopes or similar devices having relatively narrow operating portions capable of insertion through a small incision in the skin.

The aspects of the present disclosure may be modified for use with various methods for retrieving tissue specimens during a surgical procedure and, in particular, a minimally invasive surgical procedure including, for example, cholecystectomies, appendectomies, nephrectomies, colectomies, splenectomies, laparoscopic hysterectomies, and the like.

The presently disclosed specimen retrieval device will now be described in detail with reference to the drawings in which like reference numerals designate identical or corresponding elements in each of the several views. In this description, the term “proximal” is used generally to refer to that portion of the device that is closer to a clinician, while the term “distal” is used generally to refer to that portion of the device that is farther from the clinician. The term “clinician” is used generally to refer to medical personnel including doctors, surgeons, nurses, and support personnel.

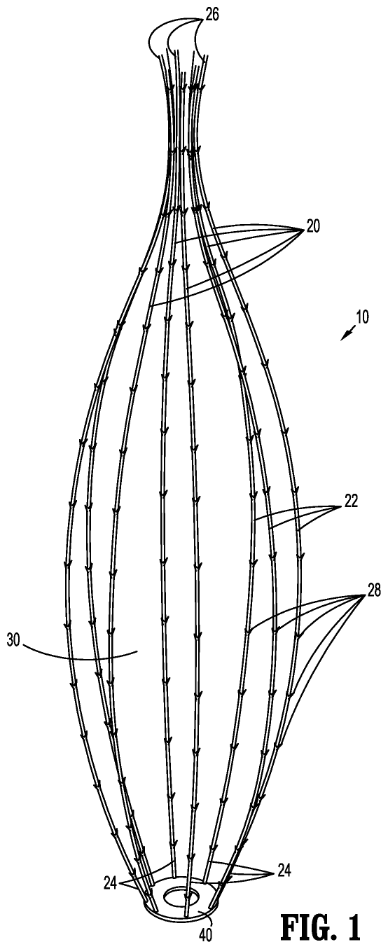

FIG. 1 illustrates a specimen retrieval device 10 according to an aspect of the disclosure. The specimen retrieval device 10 includes a plurality of barbed threads 20 and a centering ring 40. The plurality of barbed threads 20 used to form the specimen retrieval device 10 may number from about 4 to about 12 barbed threads, in aspects from about 6 to about 10 barbed threads, in other aspects about 8 barbed threads. The barbed threads 20 have an elongate body 22, a distal portion 24, a proximal portion 26, and barbs 28. As shown, the distal portions 24 of the barbed threads 20 are circumferentially affixed to the centering ring 40. The barbed threads 20 form a cage-like or whisk configuration with a space 30 formed within the circumference of the barbed threads 20.

The barbed threads 20 and the centering ring 40 of the specimen retrieval device 10 may be formed of any suitable biocompatible material. For example, suitable biocompatible materials include polymeric materials such as those derived from lactones including lactide, glycolide, caprolactone, and valerolactone; carbonates such as trimethylene carbonate, tetramethylene carbonate, and the like; dioxanones such as 1,4-dioxanone; 1,dioxepanones such as 1,4-dioxepan-2-one and 1,5-dioxepan-2-one; ethylene glycol; ethylene oxide; ester amides; γ-hydroxyvalerate; β-hydroxypropionate; alpha-hydroxy acid; hydroxybutyrates; poly (ortho esters); hydroxy alkanoates; tyrosine carbonates; polyimide carbonates; polyimino carbonates such as poly (bisphenol A-iminocarbonate) and poly (hydroquinone-iminocarbonate); polyurethanes; polyanhydrides; polybutesters; and copolymers and combinations thereof.

The barbs 28 may be formed on the barbed threads 20 by any method within the purview of those skilled in the art, including cutting or similar processes utilized for forming barbs on sutures or similar thread-like devices. In aspects, suitable barbed threads 20 utilized with the specimen retrieval device 10 include those sold commercially as V-LOC™ sutures from Medtronic (North Haven, Conn.).

The orientation of the barbs 28 is such that the tips of the barbs 28 face proximally. When the proximal portions 26 of the barbed threads 20 are proximally pulled by the clinician, the barbs 28 contact and grab a tissue specimen “TS” to be removed from the patient.

Although not shown, the proximal portions 26 of the barbed threads 20 may be looped, tied, welded, threaded through a pledget, woven through a ring or attached to a handle to aid the clinician in pulling and to prevent the barbed threads 20 from slipping into the patient's body cavity “BC”. The threads may also be separated by completely circular or incomplete spacer segments to help form the space 30 within the circumference of the barbed threads 20 for placement of a tissue specimen to be removed from a patient with the specimen retrieval device 10.

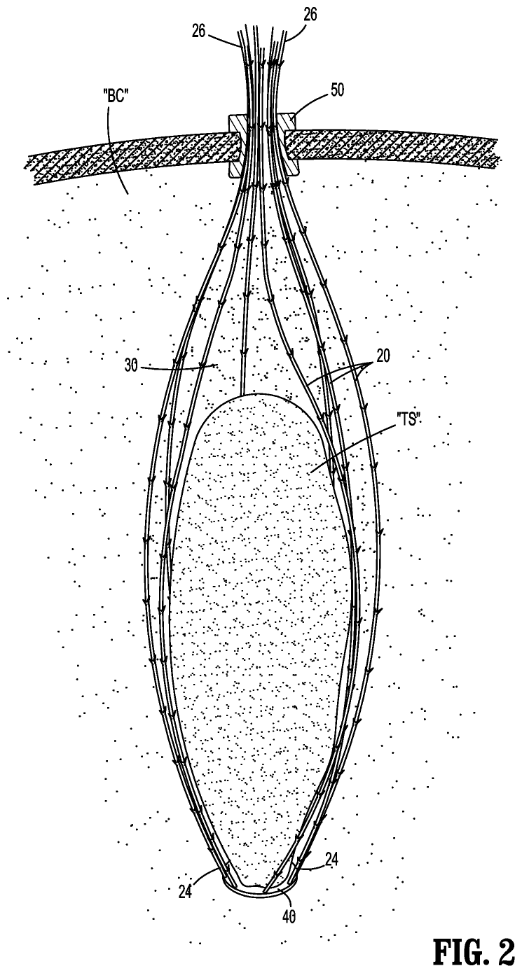

In use, as depicted in FIG. 2, the centering ring 40 and distal portions 24 of the barbed threads 20 may be introduced into a patient's body cavity “BC” through an access port 50. The barbed threads 20 are in a relaxed configuration, permitting placement of a tissue specimen “TS” within the space 30 formed within the circumference of the barbed threads 20.

After placing the tissue specimen “TS” within the space 30 formed within the circumference of the barbed threads 20, the clinician operates the device by pulling the proximal portions 26 of the barbed threads 20 in a proximal direction, sequentially or simultaneously, to reduce the volume of the space 30 within which the tissue specimen “TS” has been placed, so that the barbs 28 of the barbed threads 20 come into contact with the tissue specimen “TS”, thereby securing the tissue specimen “TS” within the space 30 formed within the circumference of the barbed threads 20. For example, the proximal portions 26 of the barbed threads 20 may be pulled in a clock-wise or counterclockwise pattern, or a cross pattern, with opposing barbed threads 20 pulled to secure the tissue specimen “TS” within the space 30 formed within the circumference of the barbed threads 20.

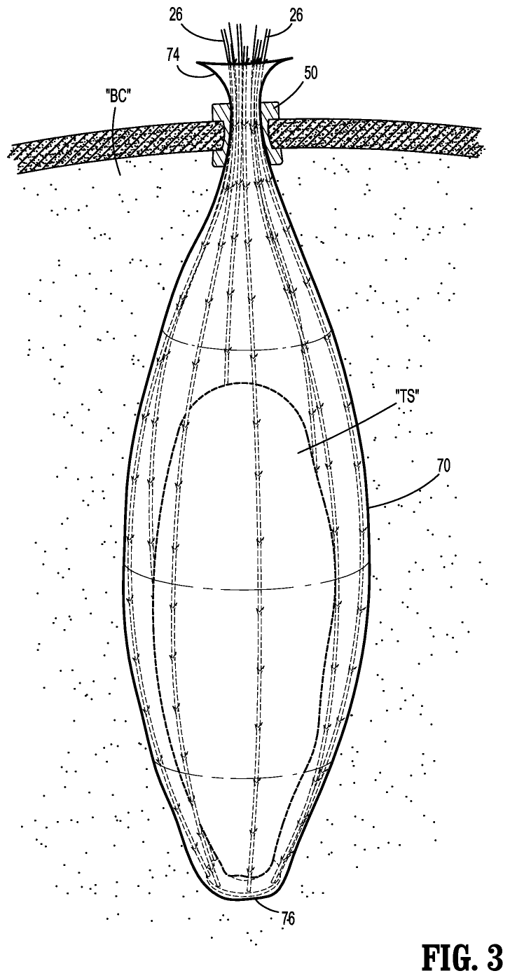

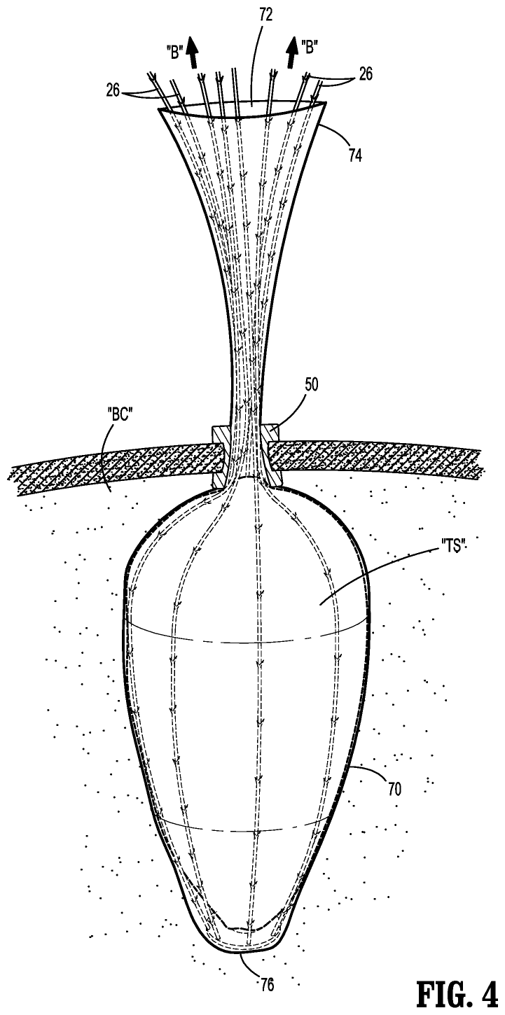

Once a tissue specimen “TS” has been secured within the space 30 formed within the circumference of the barbed threads 20, as depicted in FIGS. 3 and 4, the specimen retrieval device 10 (shown in phantom) may then be placed within a specimen bag 70 for further containment. The specimen bag 70 includes a generally tubular or elongated configuration that includes an opening 72 (see, FIG. 4) at a proximal portion 74 of the specimen bag 70 and a closed portion 76. Alternatively, other specimen bag configurations are envisioned.

The specimen bag 70 may be made from any suitable biocompatible material (e.g., nylon, urethane, ripstop nylon or latex) capable of forming a flexible collapsible member, or membrane. In aspects, the material from which the specimen bag is made is resilient, antistatic, pyrogen-free, non-toxic, and sterilizable. The specimen bag 70 may be opaque, translucent, or clear.

As shown in FIG. 3, the proximal portion 74 of the specimen bag 70 and the proximal portions 26 of the barbed threads 20 are externalized. As shown in FIG. 4, the clinician may then proximally pull the proximal portions 26 of the barbed threads 20 and the proximal portion 74 of the specimen bag 70, as indicated by arrows “B”, to remove the specimen bag 70 and the specimen retrieval device 10 within the specimen bag 70 from the patient's body cavity “BC”.

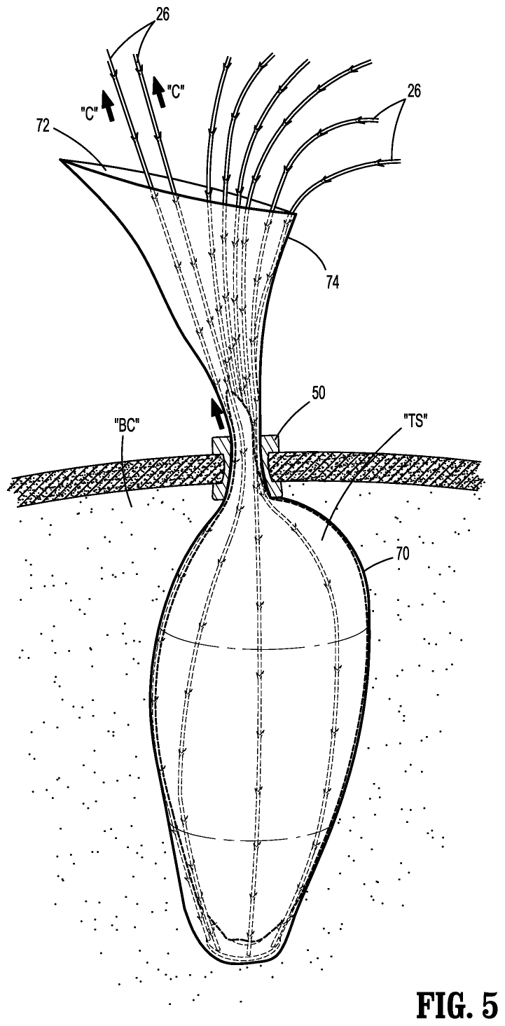

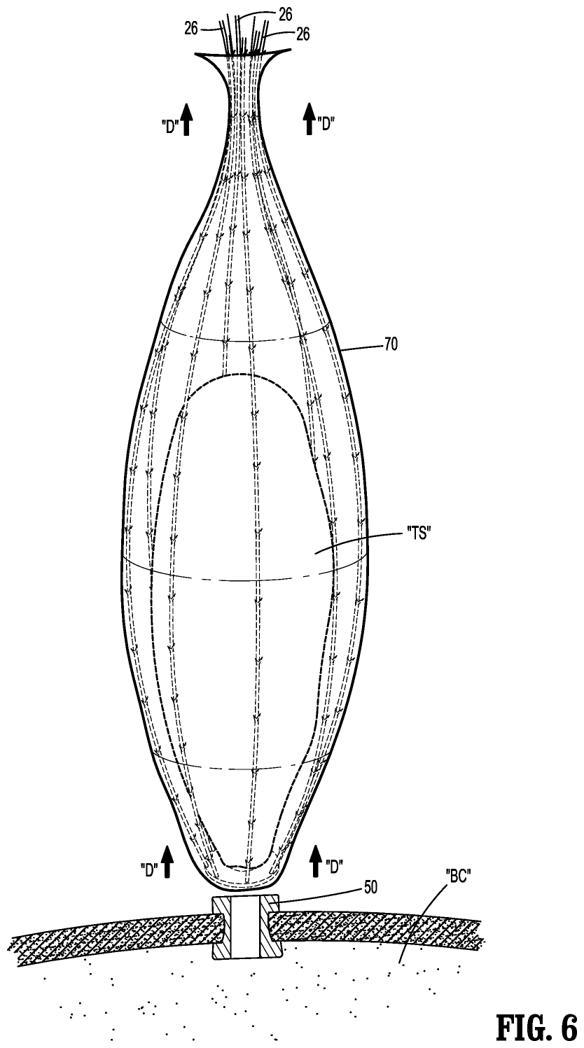

As depicted in FIG. 5, the clinician may pull the proximal portions 26 of the barbed threads 20 of the specimen retrieval device 10 on one side of the specimen retrieval device 10 (indicated by arrows “C”) to assist in advancing dense anatomy of a tissue specimen “TS” and removal of the specimen retrieval device 10 from the patient's body cavity “BC”. As depicted in FIG. 6, continued proximal pulling of the proximal portion 74 of the specimen bag 70 (indicated by arrows “D”) and proximal portions 26 of the barbed threads 20 will result in removal of the specimen retrieval device 10 and specimen bag 70 from the patient's body cavity “BC”.

Alternatively, although not depicted, in embodiments the specimen retrieval device possessing the tissue specimen “TS” may be directly removed from the patient's body, without placing the specimen retrieval device 10 within the specimen bag 70. For example, the proximal portions 26 of the barbed threads 20 may simply be continually pulled, sequentially or simultaneously, to pull the specimen retrieval device 10 containing the tissue specimen “TS” through the access port 50 or a similar opening in the abdominal wall. Once the bulk of the tissue specimen “TS” has passed through the access port 50 or other extraction site, the user may resume standard extraction techniques.

Once the specimen retrieval device of the disclosure has been removed from the patient's body, any tissue specimen may be removed from the specimen retrieval device for further examination.

Benefits of the disclosed specimen retrieval devices include their use in the removal of large tissue specimens from a body cavity. Current extraction techniques rely on pulling on a specimen or dragging an unsupported bag through an incision site. The pulling technique requires continuous grip strength when forceps are used and has the risk of tearing the specimen during extraction. The bag technique results in a large build up of tissue mass and fluid at the end of the bag, which increases in pressure until the entire bag is pulled through (or a burst occurs). The specimen retrieval device of the present disclosure allows for removal of larger tissue specimens through smaller incisions. Specimen extraction through smaller wounds provides the benefit of smaller incisions with reduced cosmetic defects.

Kits of the disclosure may include both the specimen retrieval device described above, as well as specimen bags, access ports, trocars, catheters, graspers, vacuum sources (tubes), combinations thereof, and the like.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto. Additionally, it is envisioned that the elements and features illustrated or described in connection with one exemplary aspect may be combined with the elements and features of another disclosed aspect without departing from the scope of the disclosure, and that such modifications and variations are also intended to be included within the scope of the disclosure. Accordingly, the disclosure is not to be limited by what has been particularly shown and described, except as indicated by the appended claims.

What is claimed is:

1. A specimen retrieval device comprising:
a plurality of barbed threads, each of the plurality of barbed threads having an elongate body, a distal portion, a proximal portion, and barbs extending from the elongate body; and
a centering ring,
wherein the distal portions of the plurality of barbed threads are circumferentially affixed to the centering ring.

2. The specimen retrieval device of claim 1, wherein a space is formed within a circumference of the plurality of barbed threads.

3. The specimen retrieval device of claim 1, wherein the plurality of barbed threads includes from about 4 to about 12 barbed threads.

4. The specimen retrieval device of claim 1, wherein the plurality of barbed threads includes from about 6 to about 10 barbed threads.

5. The specimen retrieval device of claim 1, wherein the plurality of barbed threads are formed of a biocompatible material selected from polymeric materials derived from lactones, carbonates, dioxanones, ethylene glycol, ethylene oxide, ester amides, γ-hydroxyvalerate, β-hydroxypropionate, alpha-hydroxy acid, hydroxybutyrates, poly (ortho esters), hydroxy alkanoates, polyurethanes, polyanhydrides, polybutesters, copolymers thereof, or combinations thereof.

6. The specimen retrieval device of claim 1, wherein the centering ring is formed of a biocompatible material selected from polymeric materials derived from lactones, carbonates, dioxanones, ethylene glycol, ethylene oxide, ester amides, γ-hydroxyvalerate, β-hydroxypropionate, alpha-hydroxy acid, hydroxybutyrates, poly (ortho esters), hydroxy alkanoates, polyurethanes, polyanhydrides, polybutesters, copolymers thereof, or combinations thereof

7. The specimen retrieval device of claim 1, wherein tips of the barbs face proximally.

8. A kit comprising:
a specimen bag; and
a specimen retrieval device comprising a plurality of barbed threads, each of the plurality of barbed threads having an elongate body, a distal portion, a proximal portion, and barbs extending from the elongate body, the specimen retrieval device also including a centering ring,
wherein the distal portions of the plurality of barbed threads are circumferentially affixed to the centering ring.

9. The kit of claim 8, wherein the specimen bag includes a tubular configuration having an opening at a proximal portion of the specimen bag and a closed portion.

10. The kit of claim 8, wherein the specimen bag is formed of a biocompatible material selected from nylon, urethane, ripstop nylon or latex.

11. The kit of claim 8, further comprising at least one additional component selected from access ports, trocars, catheters, graspers, vacuum sources, or combinations thereof.

12. The kit of claim 11, wherein the at least one additional component is an access port.

13. The kit of claim 8, wherein the plurality of barbed threads includes from about 4 to about 12 barbed threads.

14. The kit of claim 8, wherein the plurality of barbed threads includes from about 6 to about 10 barbed threads.

15. The kit of claim 8, wherein tips of the barbs face proximally.

16. A method of removing tissue from a patient comprising:
introducing a specimen retrieval device into a patient, the specimen retrieval device including a plurality of barbed threads, each of the plurality of barbed threads having an elongate body, a distal portion, a proximal portion, and barbs extending from the elongate body, and a centering ring, wherein the distal portions of the plurality of barbed threads are circumferentially affixed to the centering ring and a space is formed within a circumference of the plurality of barbed threads;
introducing a tissue specimen into the space formed within the circumference of the plurality of barbed threads;
pulling the proximal portions of the plurality of barbed threads so that the plurality of barbed threads come into contact with the tissue specimen, thereby securing the tissue specimen within the space formed within the circumference of the plurality of barbed threads; and
removing the specimen retrieval device from the patient.

17. The method of claim 16, wherein pulling the proximal portions of the plurality of barbed threads occurs by pulling the proximal portions sequentially.

18. The method of claim 16, wherein pulling the proximal portions of the plurality of barbed threads occurs by pulling the proximal portions simultaneously.

19. The method of claim 16, further comprising introducing the specimen retrieval device into the patient through an access port.

20. The method of claim 16, further comprising inserting the specimen retrieval device into a specimen bag prior to removing the specimen retrieval device from the patient.
